(19) United States
(12) Patent Application Publication
(10) Pub. No.: US 2012/0017913 A1
(43) Pub. Date: Jan. 26, 2012

(54) Tracheostomy Tube Loading Catheter

(76) Inventors: James F. Schumacher, Cumming, GA (US); Sam C. Chan, Atlanta, GA (US); Nathan C. Griffith, Roswell, GA (US); F. Anthony Headley, JR., Atlanta, GA (US)

(21) Appl. No.: 12/843,449

(22) Filed: Jul. 26, 2010

(51) Int. Cl. A61M 16/04

(52) U.S. Cl. 128/207.14

(57) ABSTRACT

There is provided a tracheostomy tube loading catheter with a handle, mid-section, tip portion and guiding catheter portion, where the guiding catheter and tip portions are non-detachably attached to the mid-section, and where the loading catheter has a cannula therethrough.

Ventilators or respirators are used for mechanical ventilation of the lungs of a patient in a medical setting. The ventilator unit is connected to a hose set; the ventilation tubing or tubing circuit, delivering the ventilation gas to the patient. At the patient end, the ventilation tubing is typically connected to a tracheal ventilation catheter or tube, granting direct and secure access to the lower airways of a patient. Tracheal catheters are equipped with an inflated sealing balloon element, or “cuff”, creating a seal between the tracheal wall and tracheal ventilation tube shaft, permitting positive pressure ventilation of the lungs.

One type of tracheal catheter, an endotracheal tube (ET tube), inserted through the mouth, is generally used for a number of days before a decision is made to switch a patient to a tracheostomy tube, inserted directly into the trachea through a stoma in the tracheal wall. Endotracheal tubes have been linked in some studies to an increased rate of ventilator acquired pneumonia (VAP) and so tracheostomy operations are becoming increasingly common and are being performed earlier in the patient's hospital stay in order to reduce the occurrence of VAP.

A tracheostomy procedure involves making a small horizontal incision in the skin of the neck to grant access to the trachea. Because of the uniquely flexible and elastic nature of the trachea, it has been found that healing is much faster if only a small hole is made in the tracheal wall and the hole dilated, rather than cutting the tracheal wall. After the skin incision, a hemostat or other implement may be used to separate the subcutaneous tissues to gain access to the trachea, and digital palpation is used to locate the tracheal rings. A bronchoscope is usually inserted into the ET tube and the tube withdrawn from the trachea until the light of the bronchoscope transdermally illuminates the site of the incision. A sheathed needle is used to puncture the tracheal wall, usually between the second and third tracheal rings. The needle is removed with the sheath remaining, a flexible guide wire (also called a J-wire) is inserted in the place of the needle and the sheath is removed. The bronchoscope is used for viewing the procedure from within the trachea in order to avoid damage to the tracheal wall. A small (e.g. 14 French) introducer dilator is introduced over the guide wire to perform an initial dilation of the tracheal wall, and then removed. A smaller (e.g. 8 French) guiding catheter is then introduced over the guide wire. (Note, French is a measure of circumference based on the theory that non-round tubes of the same circumference will fit into the same incision. One French is approximately 0.33 mm or 0.013 inch).

After the guiding catheter is introduced, a first dilator such as the Cook Medical Inc. Blue Rhino® dilator (see also U.S. Pat. No. 6,637,435), is placed over the guide wire and the guiding catheter and first dilator are advanced into the trachea through the tracheal wall as a unit to perform the dilation. Cook Medical recommends a slight over-dilation of the tracheal wall in order to make the placement of the tracheostomy tube easier. After dilation, the first dilator is removed and the tracheostomy tube (with cannula removed) is introduced over the guide catheter using a second, loading dilator that fits just inside the trachostomy tube and protrudes about 2 cm beyond the distal end of the tracheostomy tube. The guide catheter, second dilator and tracheostomy tube are advanced into the trachea through the tracheal wall as a unit. Once the tracheostomy tube is at the proper depth, the second dilator, guide catheter and guide wire are removed through the tracheostomy tube, the inner cannula inserted into the tracheostomy tube and the tube connected to the ventilator.

As can be understood from the above description, the current state of the art for tracheostomy involves numerous steps and the insertion and removal of a number of components before the successful completion of the procedure. For most of this time, the patient is disconnected from the ventilator and is therefore, not breathing. In addition, the large number of parts used in current tracheostomy kits increases the likelihood that an item may be accidentally rendered unsterile and be unable to be used. In such cases, the patient must be re-intubated with an ET tube. Even if the procedure proceeds uneventfully, however, the amount of time the patient is not breathing is significant; on the order of 7 minutes or more. This is clearly a significant event, especially for a patient who is, most likely, not in optimal physical condition.

There remains a need for a device that can more quickly and safely allow for the successful placement of a tracheostomy tube.

### SUMMARY OF THE INVENTION

There is provided a one piece tracheostomy tube loading catheter (“the device”). The device includes a loading catheter portion, a tip portion and a guiding catheter portion and has a cannula therethrough for accepting a guide wire. The device may be inserted into a tracheostomy tube, slid over the guide wire and moved into the trachea. Once the trach tube is in position, the entire device and guide wire may be withdrawn through the trach tube.

The loading catheter may be used in conjunction with a one piece dilator described in patentee's sister case “Dilator with Integrated Guiding Catheter” filed on the same day as this case. The dilator has a body portion, a tip portion and a guiding catheter portion which are non-detachably attached. The dilator also has a cannula therethrough for accepting a guide wire.

Kits may be prepared having any combination of a dilator, loading catheter and tracheostomy tube. This allows for the quick and easy selection of the proper combination of articles needed for the procedure, based on the size of the trachea of the patient.

### BRIEF DESCRIPTION OF THE DRAWINGS

FIG. 1 is a drawing of the prior art Blue Rhino® dilator.

FIG. 2 is a drawing of the dilator with integrated guiding catheter.

FIG. 3 is a drawing of the dilator with integrated guiding catheter being moved into the trachea through the tracheal wall.

FIG. 4 is a drawing of the tracheostomy tube loading catheter 50.

FIG. 5 is a drawing of the trachestomy tube 26 showing the flange for attachment to the throat and shown with the cannula removed.

FIG. 6 is a drawing of the loading catheter 50 installed in the trach tube 26.

FIG. 7 is a drawing of the tracheostomy tube 26 and loading catheter 50 that have been passed over guide wire 16 and partially into the trachea 24.

FIG. 8 is a drawing of the position of the tube 26 and loading catheter 50 as they are passed further (about half way) into the trachea 24 as a unit.

FIG. 9 is a drawing of the tracheostomy tube 26 fully inserted into the trachea 24 with the flange 70 against the throat, and the loading catheter 50 and guide wire 16 being withdrawn through the tracheostomy tube 26, with the tube 26 remaining in place in the trachea 24.

FIG. 10 is a drawing of the trach tube in its final position in the trachea, with the loading catheter 50 and guide wire 16 withdrawn and the trach tube cuff 30 inflated.

FIG. 11 is a drawing of the replaceable (disposable) cannula 80 for use with the trach tube 26.

FIG. 12 is a drawing of the trach tube 26 showing the removable cannula 80 installed in the tube 26.

### DETAILED DESCRIPTION OF THE INVENTION

Tracheostomy is a lifesaving procedure to allow a patient to be ventilated directly through the trachea. Tracheostomy is also believed by many to prevent or retard the onset of ventilator acquired pneumonia (VAP). This lifesaving procedure, unfortunately, is relatively time consuming and current technology requires a large number of steps and pieces of equipment that must remain sterile and functioning properly in order to arrive at a successful conclusion. The tracheostomy procedure may be greatly improved using the loading catheter described in the Summary above in conjunction with the Dilator with Integrated Guiding Catheter.

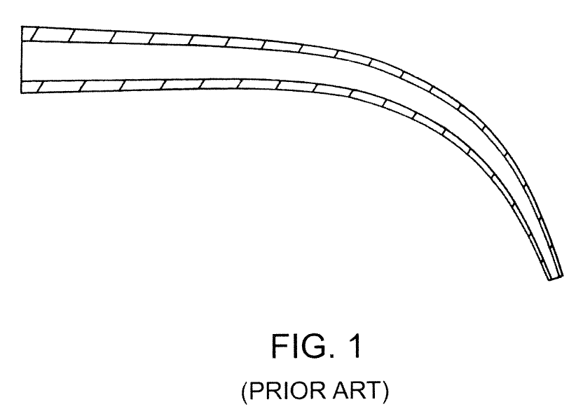

Dilators are instruments or substances for enlarging a canal, cavity, blood vessel or opening, according to the American Heritage Stedman's Medical dictionary 2001. FIG. 1 is a drawing of the prior art dilator from Cook Medical Inc. known as the Blue Rhino® dilator (see also U.S. Pat. No. 6,637,435). The '435 patent describes a one piece dilator having a generally linear shaft and a short distal tip portion with a curved tapered portion in between.

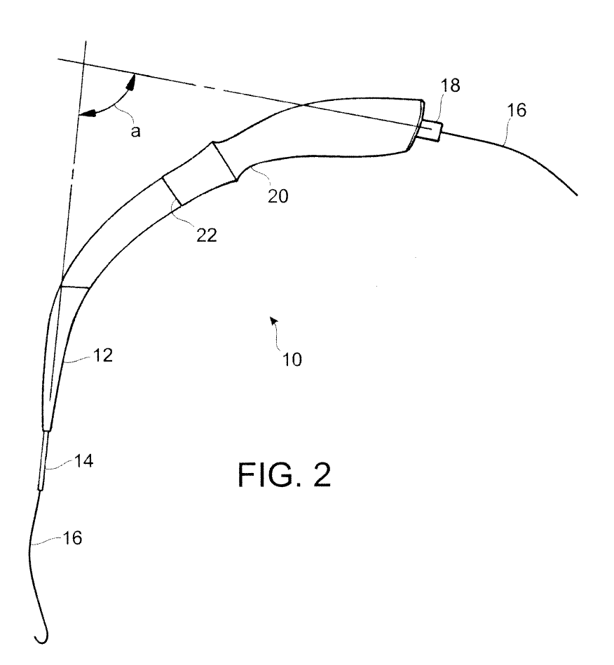

Turning to the Figures, one embodiment of a dilator 10 has a body 20 portion, a distal tip 12 portion and a guiding catheter 14 portion (FIG. 2). The dilator 10 is a single part or piece wherein the tip 12 is non-detachably attached to the body 20. The tip 12 is also desirably non-detachably attached to a guiding catheter 14 and is desirably tapered. The body 20 has a marking line 22 or alternatively a ridge where the diameter is approximately 42 French which serves as a depth marking or insertion stopping point for the dilation procedure. The body 20 has a distal portion 44 and a handle portion 46. Guide wires are generally between about 0.2 and 0.5 mm in diameter and the guiding catheter should be just slightly larger than the guide wire, or about 8 to 11 French. It should be noted that, although the dilator is described as having non-detachable body, tip and guiding catheter portions, implying separate pieces, one single piece dilator could be made, having approximately the same dimensions as the three portions. Further, a dilator having a non-detachable tip and detachable guiding catheter may be used, but is less desirable.

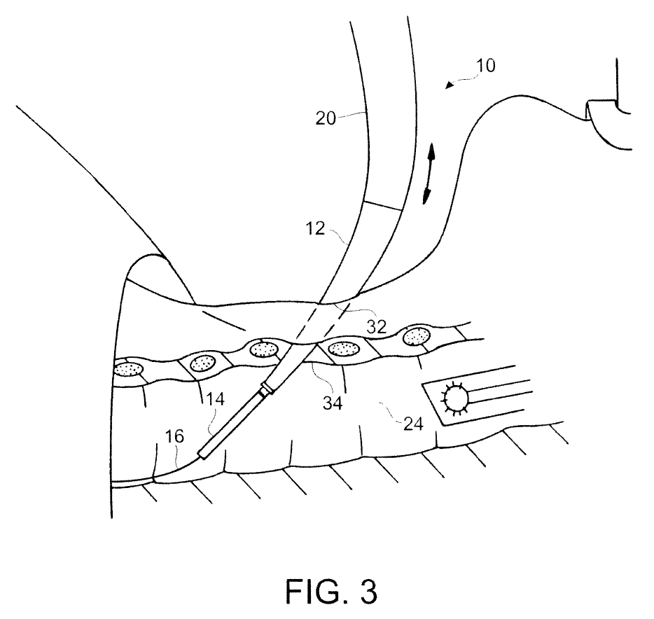

As described above, once the guide wire 16 is inserted into the trachea 24 through the incision 32 and tracheal wall 34, the dilator with integrated guiding catheter 10 may be introduced over the guide wire 16. The dilator 10 is then moved into the trachea 24 through the tracheal wall 34 until the marking line 22 of the dilator 10, which serves as a “stop” mark or depth gauge, meets the incision 32 in the throat (FIG. 3). The actual procedure of dilation of the tracheal wall involves the repeated incremental insertion and removal of the dilator 10. This procedure may be made easier for the medical provider and less traumatic for the patient by the application of a lubricious coating to the dilator 10. The coating can reduce friction and drag on the guide wire 16 and also reduce trauma to the area of the incision 32 and the tracheal wall 34. The coating may be for example, a poly(N-vinyl) lactam such as those available from Hydromer Inc., 35 Industrial Parkway, Branchburg, N.J. and as described in U.S. Pat. Nos. 5,156,601, 5,258,421, 5,420,197 and 6,054,504. The dilator may be dipped in water just before the guide wire is inserted and may be coated on the inside and/or outside. An inside coating allows the guide wire to slip through the interior of the dilator quite easily and the exterior coating avoids trauma to the skin or trachea.

Once the trachea 24 is satisfactorily dilated, the dilator 10 may be removed from the trachea 24, leaving only the guide wire 16.

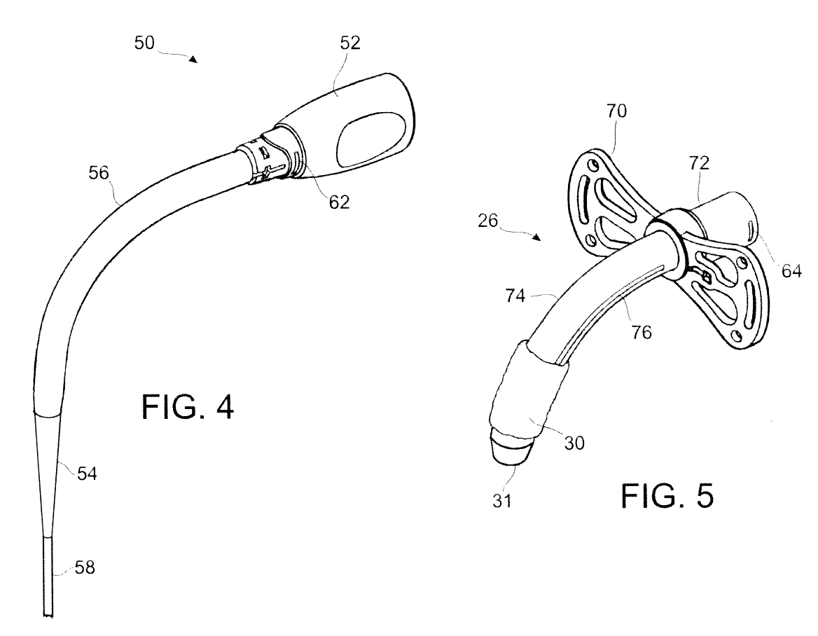

FIG. 4 shows the loading catheter 50. The loading catheter has a desirably freely rotating handle 52 at the proximal end and a non-detachably attached tip 54 and non-detachably attached guiding catheter 58 at the distal end. The guiding catheter 58 may extend proximally (not shown) through the midsection 56 and out of the handle 52 if desired. The handle 52 need not be able to rotate an entire 360 degrees but is should move sufficiently to disengage the lock mechanism used to attach the loading catheter 50 to the trach tube 26, as discussed below. The midsection 56 (between the handle 52 and tip 54) may be tubular and is flexible so that it can bend as it is inserted and removed from the trach tube 26. Suitable materials for the midsection 54 are thermoplastic elastomers, rubbers or flexible or softer plastics like polyurethanes and some polyolefins. Suitable materials for the tip 54 and handle 52 are somewhat harder plastics like nylons and some polyolefins. The device must be biocompatible, free of di(2-ethylhexyl)phthalate (DEHP) and preferably free of animal derived products. Polyvinyl chloride may also be used to fabricate the components.

The loading catheter described herein and the dilator with integrated guiding catheter may be used separately or with other dilators and trach tubes but are preferably are used together. This is preferred since this makes problem dilations easier and quicker to address and because the number of parts is greatly reduced. For example, if it becomes necessary during a procedure to stop the insertion of a trach tube and re-dilate the trachea, using the loading catheter described herein will allow its quick removal. And using the dilator with guiding catheter will allow for its quick insertion. In contrast, using other dilators and loading catheters, generally with a larger number of removable parts (e.g. a guiding catheter) requires great dexterity and a high degree of attention and concentration from the physician so that the guiding catheter or other parts are not misplaced or forgotten entirely. It is also possible that a separate guiding catheter may move into the tip of the dilator of loading catheter during a procedure and this risk becomes greater in a troubled procedure and becomes another source of distraction for the physician. Freeing the physician to concentrate only on the dilation and placement of the trach tube, without worrying about the movement or loss of parts, should improve the success rate of this procedure.

It is also desirable that the integrated guiding catheter 14 and tip 12 of the dilator 10 be sized to match the guiding catheter 58 and tip 54 of the loading catheter 50. This helps ensure that the loading catheter 50 and trach tube 26 will move smoothly into the stoma that has been dilated with the dilator 10. If the same dimensions are used on the dilator tip 12 and the loading catheter tip 54 as well as the two guiding catheters 14, 58, the possibility of a mis-fitting or difficult tube placement is greatly reduced. By “matching” the dilator to the loading catheter, what is meant is that the dimensions of the dilator and loading catheter are nearly identical; the outer dimensions of the dilator tip and guiding catheter portions are desirably within 10 percent of the outer dimensions of the loading catheter tip and guiding catheter portions or, still more desirably, the outer dimensions of the dilator tip and guiding catheter portions are desirably within 5 percent of the outer dimensions of the loading catheter tip and guiding catheter portions.

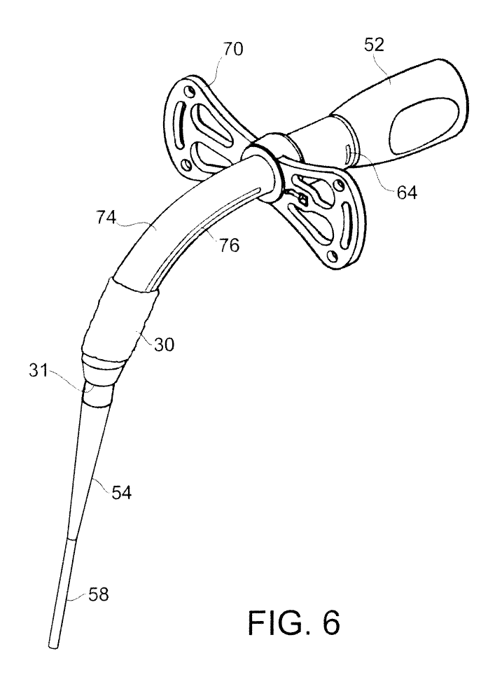

It is also desirable that the loading catheter be matched with the appropriately sized tracheostomy tube. Trach tubes are available in varying sizes, having different diameter inner cannulas, since patients have varying size tracheas. Currently available loading catheters are generally sold separately from trach tubes in kits containing multiple loading catheters, and so a selection of the proper loading catheter must be made by the physician, in order to match the trach tube needed for the patient. Matching the outer diameter of the loading catheter to the inner diameter of the trach tube and making this available together in a kit would make the selection process easier since only a trach tube size selection would need to be made. This single selection is a selection the physician is already making, so a matched loading catheter and trach tube represent a time savings and simplification of the procedure. It is also desirable to package matched tracheostomy tubes and loading catheters in a “pre-loaded” condition, meaning the loading catheter is inserted into the tracheostomy tube in a ready-to-be-used arrangement as shown in FIG. 6. Upon opening a pre-loaded kit, the physician need only remove the nested trach tube and loading catheter and they are immediately ready for insertion over the guide wire. By “matching” a loading catheter to a trach tube, what is meant is that the loading catheter fits snugly within the trach tube so that the proximal end of the tip portion of the loading catheter provides a smooth transition to the trach tube; i.e., the outer diameter of the tip is just slightly less than the inner diameter of the trach tube distal end. More desirably, the outer diameter of the proximal end of the tip portion is within 10 percent of the inner diameter of the trach tube, still more desirably the outer diameter of the proximal end of the tip portion is within 5 percent of the inner diameter of the trach tube.

Kits for the tracheostomy procedure may be prepared containing any combination of the dilator with integrated guiding catheter, the loading catheter and the tracheostomy tube discussed herein. This allows for the quick and easy selection of the proper combination of articles needed for the procedure, based on the size of the trachea of the patient.

An exemplary tracheostomy tube is shown in FIG. 5. There is a flange 70 on the trach tube 26 on the proximal end that is used to attach the trach tube to a patient's throat. The flange 70 extends on either side of the tube 26 near the proximal end where the ventilator connection 72 is located. The flange 70 is flexible and non-irritating and can be sutured onto the throat of a patient to anchor the tube 26. The size of the flange will vary depending on the size and needs of the patient. The tube 26 also has a hollow shaft 74 extending from the proximal end to the distal end 31. An inflation line 76 runs from the proximal end to the balloon cuff 30 so that the cuff may be inflated to obdurate the trachea.

In use, the loading catheter 50 is slid into the tracheostomy tube 26 (FIG. 6). The loading catheter handle 52 may detachably engage the proximal end of the trach tube 26 with, for example, a slot 64 and tab 62 arrangement as shown in FIGS. 4 and 5 where there are tabs 62 on both sides of the handle 52 which mate with slots 64 on the proximal end of the trach tube 26. Once engaged, the handle is desirably not freely rotatable. Those skilled in the art may easily devise alternative ways of mating the handle 52 with the tube 26.

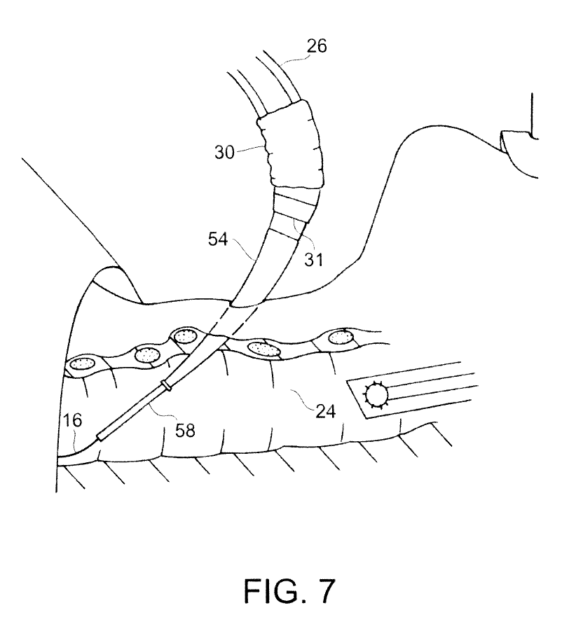

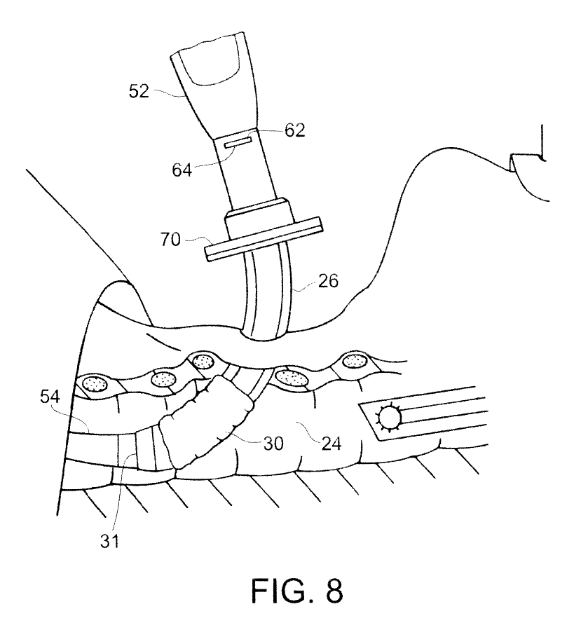

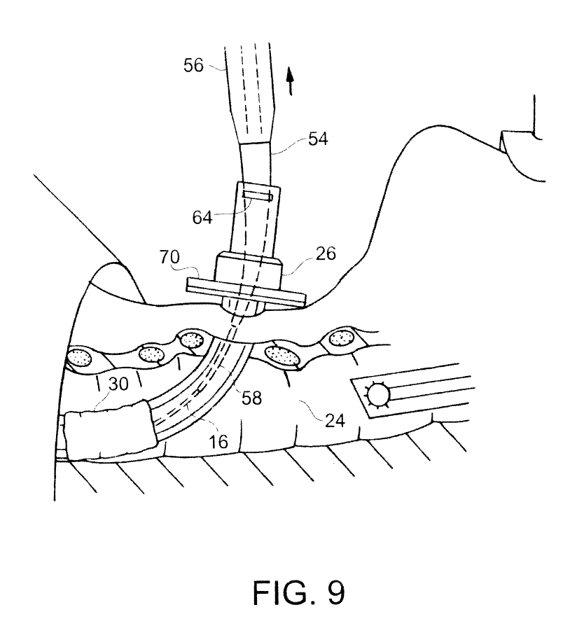

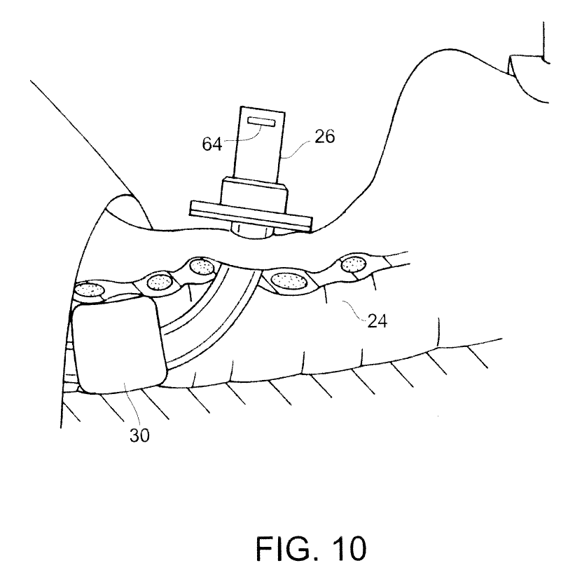

The tracheostomy tube 26 with the loading catheter 50 inserted (FIG. 6) is then axially passed over the guide wire 16. The loading catheter 50 and tube 26 are then passed into the trachea 24 as a unit (FIGS. 7 and 8 sequentially) to the point where the flange 70 on the tube 26 reaches the throat (FIG. 9). Once the flange 70 reaches the throat the tube 26 is in place in the trachea 24. The loading catheter 50 (with the non-detachably attached tip 54 and guiding catheter 58) and the guide wire 16 may be withdrawn through the tracheostomy tube 26 (FIG. 9) with only the tube 26 remaining in place in the trachea 24 (FIG. 10). Once the trach tube 26 is in place, the tube cuff 30 is inflated (FIG. 10) and the tube 26 is connected to a ventilator (not shown) and placed in service.

The loading catheter 50 may be withdrawn from the tube 26 by disengaging the detachably attached handle 52 from the proximal end of the tracheostomy tube 26 and pulling the handle 52 away from the tube 26. One way of accomplishing this disengagement is by twisting the loading catheter handle 52. This twisting action cams the loading catheter handle 52 off the proximal end of the trach tube 26, overcoming any static friction that may exist in the system and defeating the tabs 62 and slots 64 locking the loading catheter handle 52 to the tube 26. This action allows the user to pull all the loading components out through the inner lumen of the trach tube 26, leaving only the tube 26 in place.

The trach tube 26 has a balloon cuff 30 around its circumference on a lower (distal) portion of the tube that serves to block the normal air flow in the trachea so that (assisted) breathing takes place through the trach tube using a ventilator. The cuff is desirably made from a soft, pliable polymer such as polyurethane, polyethylene teraphihalate (PETP), low-density polyethylene (LDPE), polyvinyl chloride (PVC), polyurethane (PU) or polyolefin. It should be very thin; on the order of 25 microns or less, e.g. 20 microns, 15 microns, 10 microns or even as low as 5 microns in thickness. The cuff should also desirably be a low pressure cuff operating at about 30 mmH₂O or less, such as 25 mmH₂O, 20 mmH₂O, 15 mmH₂O or less. Such a cuff is described in U.S. Pat. No. 6,802,317 which describes a cuff for obturating a patient's trachea as hermetically as possible, comprising: a cuffed balloon which blocks the trachea below a patient's glottis, an air tube, the cuffed balloon being attached to the air tube and being sized to be larger than a tracheal diameter when in a fully inflated state and being made of a soft, flexible foil material that forms at least one draped fold in the cuffed balloon when inflated in the patient's trachea, wherein the foil has a wall thickness below or equal to 0.01 mm and the at least one draped fold has a loop found at a dead end of the at least one draped fold, that loop having a small diameter which inhibits a free flow of secretions through the loop of the at least one draped fold. Another description of such a cuff is in U.S. Pat. No. 6,526,977 which teaches a dilator for obturating a patient's trachea as hermetically as possible, comprising a cuffed balloon which blocks the trachea below a patient's glottis, an air tube, the cuffed balloon being attached to the air tube and being sized to be larger than a tracheal diameter when in a fully inflated state and being made of a sufficiently soft, flexible foil material that forms at least one draped fold in the cuffed balloon when fully inflated in the patient's trachea, wherein the at least one draped fold formed has a capillary size which arrests free flow of secretions across the balloon by virtue of capillary forces formed within the fold to prevent aspiration of the secretions and subsequent infections related to secretion aspiration.

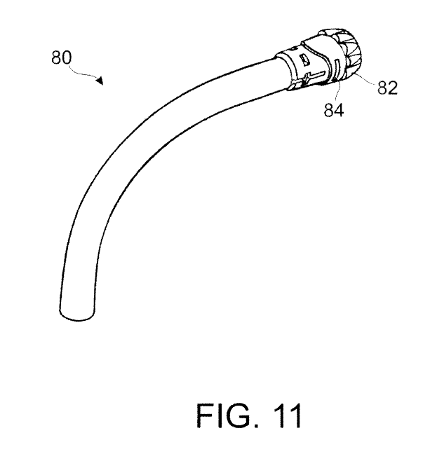

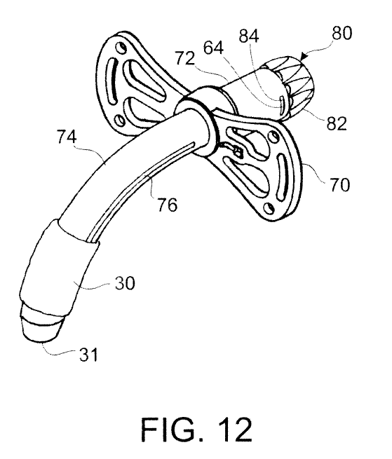

The trach tube 26 also may be used with disposable cannulas 80 (FIG. 11) that are placed within the trach tube from the proximal end (FIG. 12) These disposable cannulas 80 are changed regularly so that bacterial growth is kept to a minimum. The cannulas are made from a plastic material such as a polyolefin, polyurethane, nylon, etc and are desirably flexible. Cannulas may be treated with anti-bacterial and/or anti-viral coatings or other active materials to help reduce the growth of harmful organisms. The cannula 80 may be attached to the trach tube 26 in a manner similar to the attachment of the loading catheter 50, i.e., using tabs 84 that mate with the slots 64 on the tube exposing only the cannula end 82 on the proximal end. The cannula distal end is either flush with the trach tube distal end 31 or extends a very short distance beyond.

Exemplary sizes for the various components of the dilator and loading catheter are as follows;

The dilator body 20 portion, for example, should have a total length of less than 28 cm. The dilator tip 12 portion may be between about 25 and 80 mm in length, particularly about 35 mm long, tapering from 3 to 6 mm at the distal end to about 5 to 16 mm, particularly 4 mm at the distal end to 8 mm. The guiding catheter 14 portion may be between 1 and 5 cm in length.

The distance from the flange 70 to the distal tip 31 of the trach tube 26 may be an arched distance of between 70 and 100 mm, desirably between about 75 and 95 mm and more desirably between 80 and 90 mm. The angle of the trach tube from the flange to the distal end is between 85 and 120 degrees, desirably between 95 and 115 degrees, more desirably between 100 and 110 degrees. The flange 70 may desirably be of a width between 6 and 12 cm and height of 1 to 6 cm, more particularly between 7 and 10 cm and 2 and 5 cm respectively or still more particularly between 8 and 9 cm and 2 and 4 cm respectively.

The loading catheter 50 has a desirably tubular midsection having an arched length between about 8 and 13 cm, particularly about 11 cm and may terminate as much as 20 mm beyond the distal tip of the trach tube or may terminate within it. The handle 52 may be between 2 and 7 cm long, particularly about 5 cm. The loading catheter tip 54 portion may be between about 25 and 80 mm in length, particularly about 35 mm long, tapering from 3 to 6 mm at the distal end to about 5 to 16 mm, particularly 4 mm at the distal end to 8 mm. The guiding catheter 58 portion may be between 1 and 5 cm in length.

This application is one of two commonly assigned patent applications which are being filed on the same day. The group includes application Ser. No. ______: (attorney docket no. 64676190US01) in the name of James F. Schumacher and is entitled “Dilator with Integrated Guiding Catheter”.

As will be appreciated by those skilled in the art, changes and variations to the invention are considered to be within the ability of those skilled in the art. Such changes and variations are intended by the inventors to be within the scope of the invention. It is also to be understood that the scope of the present invention is not to be interpreted as limited to the specific embodiments disclosed herein, but only in accordance with the appended claims when read in light of the foregoing disclosure.

What is claimed is:

1. A tracheostomy tube loading catheter comprising a handle, mid-section, tip portion and guiding catheter portion, wherein the guiding catheter and tip portions are non-detachably attached to the mid-section, and wherein said loading catheter has a cannula therethrough.

2. The device of claim 1 wherein said proximal handle detachably attaches to a proximal end of said tracheostomy tube.

3. The device of claim 2 wherein said handle attaches to said tube with a tab and slot lock.

4. The device of claim 1 wherein said mid-section is as flexible or more flexible than said tip.

5. The device of claim 4 wherein said mid-section is made of a thermoplastic elastomer, rubber or flexible plastic.

6. A kit for performing a tracheostomy, said kit containing a loading catheter having non-detachably attached tip and guiding catheter portions, said kit also containing a tracheostomy tube, wherein said loading catheter matches said tracheostomy tube.

7. The kit for performing a tracheostomy of claim 6 wherein said loading catheter is pre-loaded into said tracheostomy tube.

8. The kit for performing a tracheostomy of claim 6, further comprising a dilator.

9. A kit for performing a tracheostomy, said kit containing a dilator a having non-detachably attached tip portion and having dimensions, said kit further containing a loading catheter having non-detachably attached tip and guiding catheter portions having dimensions within 10 percent of the dimensions of the non-detachably attached tip portion of the dilator.

10. The kit for performing a tracheostomy of claim 9, wherein the dimensions of said dilator tip and guiding catheter portions are within 5 percent of the dimensions of the non-detachably attached tip and guiding catheter portions of the loading catheter.

11. The kit for performing a tracheostomy of claim 9, further comprising a tracheostomy tube.

12. A method of performing a tracheostomy, comprising the steps of:
creating a stoma in the tracheal wall and inserting a guide wire, dilating said stoma with a dilator having non-detachably attached tip and guiding catheter portions, removing said dilator, inserting a tracheal tube on a loading catheter having non-detachably attached tip and guiding catheter portions over said guide wire and into said stoma until a flange on said tracheal tube contacts a throat of a patient, and removing said loading catheter and guide wire from said tracheal tube.
